(19) United States
(12) Patent Application Publication
(10) Pub. No.: US 2026/0241196 A1
(43) Pub. Date: Aug. 20, 2026

(54) INTERFACE MEDIUM FOR DERMATOLOGICAL LASER TREATMENTS

(71) Applicant: Avava, Inc., Waltham, MA (US)

(72) Inventors: Jayant Bhawalkar, Auburndale, MA (US); Karine Nadau-Fourcade, Villenueve-Loubet (FR); Emmanuelle Gutierrez, Saint Laurent du Var (FR)

(21) Appl. No.: 19/474,692

(22) PCT Filed: Apr. 3, 2024
(86) PCT No.: PCT/US2024/022825

§ 371 Date: Oct. 10, 2025

Related U.S. Application Data
(60) Provisional application No. 63/495,347, filed on Apr. 11, 2023.

(51) Int. Cl. A61N 5/06; A61K 8/34; A61N 5/067; A61Q 19/00

(52) U.S. Cl. CPC A61N 5/0616; A61K 8/34; A61N 5/067; A61Q 19/00

(57) ABSTRACT

Exemplary compositions and methods can be used for dermatological laser treatments, by providing suitable interface media between the skin and a source of electromagnetic radiation. The interface medium should act as a refractive index matching agent between the applicator window and the skin, and also reduce the friction between the applicator window and the skin so that the applicator can glide easily on the skin. The optical interface medium exhibits photostability such that optical clarity of the medium is maintained upon exposure to electromagnetic radiation without plasma formation, cavitation, or burning.

### CROSS-REFERENCE TO RELATED APPLICATIONS

This application claims the benefit of U.S. Provisional Application No. 63/495,347, filed on Apr. 11, 2023, and entitled “Interface Medium for Dermatological Laser Treatments.” The entire contents of which is hereby incorporated herein by reference in its entirety.

### BACKGROUND

Lasers and light-based therapeutic devices are widely used in the field of dermatology. Optical energy (electromagnetic radiation, or “EMR”) of appropriate wavelength, pulse duration, and intensity can alter tissue, or chromophores present in tissue through a number of different mechanisms including photo-thermal and photochemical mechanisms. These alterations have been harnessed for some of the best-known laser/light-based procedures such as laser hair removal, tattoo removal, removal of pigmented lesions and vascular lesions, tissue tightening, reduction in wrinkles, etc. Devices that are used to perform these treatments include applicators that are manually positioned and moved over the patient's skin by the treating physician. Therapeutic energy delivery through these applicators often occurs through a patient-contacting-surface such as a transparent window. A fluid may be used as the interface between the window and the skin to act as a refractive index matching agent between the applicator window and the skin, and to reduce the friction between the applicator window and the skin so that the applicator can glide easily on the skin. Many of the formulations designed to be applied to skin are unable to withstand high peak power laser pulses and can result in plasma formation, cavitation, or degradation of the formulation, which can cause skin damage or damage to the applicator window. Further, many formulations trap air bubbles when the device window glides over the skin, thereby decreasing optical transmittance through the formulation. It is therefore desirable that compositions and methods be made available that address these limitations and challenges.

### SUMMARY

Embodiments for compositions and methods for dermatological laser treatment media are described. In an embodiment, a composition is provided, including an optical interface medium for dermatological phototherapy. The optical interface medium is a single-phase, transparent, aqueous gel that is optically clear such that across a wavelength range of about 380 nm to about 1900 nm: absorbance is below 15 cm⁻¹; and optical scattering is below 1 cm⁻¹. The optical interface medium has a refractive index in the range of about 1.33 to about 1.5 with a viscosity in the range of about 60000 to about 100000 cP. The optical interface medium exhibits photostability such that optical clarity is maintained upon exposure to electromagnetic radiation up to about 10⁸ W/cm² without plasma formation, cavitation, or burning.

The composition can vary in a number of ways. For example, the optical interface medium can include 1,3-propanediol at a range of about 5 to about 70 weight percent. The optical interface medium may also include: water, at a range of about 28.5 to about 93.5 weight percent; a water-soluble refractive index adjusting agent, at a range of about A to about B weight percent; and a gelling agent, at a range of about A to about B weight percent. In some variations, the viscosity of the optical interface medium is in the range of about 60000 to about 100000 Cp (Brookfield method: LV, spindle 16, speed 1 rpm) and in some embodiments it is in the range of about 5000 to about 8000 cP (Brookfield method: RV spindle 27, speed 30 rpm). In other variations the pH of the optical interface medium is in the range of about 5 to about 6. The optical interface medium can include a numbing agent or a soothing agent.

In another embodiment, an optical interface medium for dermatological phototherapy is provided, including: water; 1,3-propanediol as a water-soluble refractive index adjusting agent; and a gelling agent, configured to form a single-phase transparent gel with water and the refractive index adjusting agent. The optical interface medium is optically clear, has a refractive index in the range of about 1.33 to about 1.43, and exhibits photostability upon exposure to electromagnetic radiation up to about 10⁸ W/cm² without plasma formation, cavitation, or burning.

The composition can vary in a number of ways, For example, the gelling agent can be a hydroxyethyl acrylate/sodium acryloyldimethyl taurate copolymer, a polyacrylate crosspolymer-6, an acrylic acid copolymer, an acrylates/C10-30 alkyl acrylate crosspolymer, a carbomer, or a sodium polyacrylate. The optical interface medium can further include one or more antioxidants, antimicrobials, soothing agents, fragrance masking agents, or preservatives. In some variations, the optical interface medium includes about 28.5 to about 93.5 percent water by weight. In some variations, the optical interface medium includes about 5 to about 70 percent refractive index adjusting agent by weight. In some variations, the optical interface medium includes about 0.05 to about 3 percent gelling agent by weight.

In another embodiment, a method is provided. The method can include applying electromagnetic radiation (EMR) to skin tissue by: applying a coupling medium to a target region of the skin; positioning an EMR delivery device in contact with the coupling medium overlaying the target region while moving the device over the skin without bubble formation within the coupling medium; and applying EMR through the device as it is moved over the target region while the coupling medium maintains photostability without exhibiting plasma formation, cavitation, or burning. In some variations, the coupling medium is optically clear with a refractive index in the range of about 1.33 to about 1.43, and a viscosity in the range of about 60000-100000 Cp (Brookfield method: LV, spindle 16, speed 1 rpm) and in some embodiments it is in the range of about 5000 to about 8000 cP (Brookfield method: RV spindle 27, speed 30 rpm). In some variations, the coupling medium exhibits photostability upon exposure to electromagnetic radiation up to about 10⁸ W/cm² without plasma formation, cavitation, or burning. In some variations, the coupling medium maintains photostability to retain optical clarity such that across a wavelength range of about 380 to about 1600 nm: absorbance is below 15 cm⁻¹; and optical scattering is below 1 cm⁻¹.

### BRIEF DESCRIPTION OF THE DRAWINGS

Further objects, features and advantages of the present disclosure will become apparent from the following detailed description taken in conjunction with the accompanying figures showing illustrative embodiments, results and/or features of the exemplary embodiments of the present disclosure, in which:

FIG. 1 is a flowchart of an example scale up manufacturing process for an interface medium for dermatological laser treatments.

FIG. 2 is a table of qualitative and quantitative data for the exemplary composition of Example A (manufacturers, suppliers, cosmetic functions and rational for using in the formula).

FIG. 3 is a table with data on functions and regulatory aspects of raw materials used in Example A

FIG. 4 is a table providing initial characterization data with preliminary specification parameters for a 600 g lab batch of Example A.1.

FIG. 5 is a table of preliminary physical stability data for Example A.1, showing that the formulation demonstrated acceptable stability characteristics after storage for 1 month at room temperature and at 40° C.

FIG. 6 is a table of initial characterization data with preliminary specification parameters for a 10 kg batch of Example A.2.

FIG. 7 is a table of data from a three month stability study of Example A.2.

FIG. 8 is a flowchart of an exemplary heating process for manufacturing of a lab batch.

FIG. 9 is a table of data from a one month stability study of Example A.4. It is noted that the drawings are not necessarily to scale. The drawings are intended to depict only typical aspects of the subject matter disclosed herein, and therefore should not be considered as limiting the scope of the disclosure. The systems, compositions, and methods specifically described herein and illustrated in the accompanying drawings are non-limiting exemplary embodiments.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

Embodiments of the disclosure are discussed in detail below with respect to fractionated treatment including skin rejuvenation and skin resurfacing, for example skin resurfacing for: acne, chickenpox and surgical scars, periorbital and perioral wrinkles, photoageing changes, facial dyschromias, and stretch marks. Additional treatments related to the disclosure include treatment of pigmentary conditions of the skin, such as melasma, and other pigmentary conditions, such as granuloma annulare.

The disclosed embodiments can be employed for treatment of other pigmentary and non-pigmentary conditions and other tissue and non-tissue targets without limit. Examples of pigmentary conditions can include, but are not limited to, post inflammatory hyperpigmentation (PIH), dark skin surrounding eyes, dark eyes, café au lait patches, Becker's nevi, Nevus of Ota, congenital melanocytic nevi, ephelides (freckles) and lentigo. Additional examples of pigmented tissues and structures that can be treated include, but are not limited to, hemosiderin rich structures, pigmented gallstones, tattoo-containing tissues, and lutein, zeaxanthin, rhodopsin, carotenoid, biliverdin, bilirubin and hemoglobin rich structures. Examples of targets for the treatment of non-pigmented structures, tissues and conditions can include, but are not limited to, hair follicles, hair shafts, vascular lesions, infectious conditions, sebaceous glands, acne, and the like.

Methods of treating various skin conditions, such as for cosmetic purposes, can be carried out using the systems described herein. It is understood that, although such methods can be conducted by a physician, non-physicians, such as aestheticians and other suitably trained personnel may use the systems described herein to treat various skin conditions with and without the supervision of a physician.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

Dermatological laser treatment requires light transmission between an EMR applicator and a skin surface. Use of an optical interface medium at the interface can help to match refractive indices of the applicator window and the skin, and to reduce the friction between the applicator window and the skin so that the applicator can glide easily on the skin. In an embodiment, the optical interface medium is optically clear across a wavelength range of interest, so as to maximize efficiency of transmission through the medium. It is advantageous for the optical interface medium to be able to withstand high peak power laser pulses without plasma formation, cavitation, or degradation of the formulation, which can cause skin damage or damage to the applicator. Further, ideal formulations should not trap air bubbles when the device window glides over the skin, as bubbles could decrease optical transmittance through the formulation.

An exemplary optical interface medium for dermatological phototherapy is a single-phase, transparent, aqueous gel. A single-phase medium avoids the potential for light scattering at phase interfaces within the medium and provides homogenous optical properties. Transparency and optical clarity provide efficient optical transmittance through the formulation. An aqueous or water-based gel can provide appropriate viscosity for use while also allowing for easy removal and cleanup. Aqueous formulations can also provide superior thermal transfer and cooling effects in comparison to non-aqueous formulations. In some implementations, a water-soluble refractive index adjusting agent is present at a range sufficient to cause the refractive index of the interface medium to be within a desired range. In various implementations, the weight percent range of the water-soluble refractive index adjusting agent is about 5-90, about 5-10, about 10-15, about 15-20, about 20-25, about 25-30, about 30-35, about 35-40, about 40-45, about 45-50, about 50-55, about 55-60, about 65-70, about 75-80, about 80-85, about 85-90, above about 90, or any combination of these ranges. Similarly, in various implementations the weight percent range of water in the interface medium is about 5-10, about 10-15, about 15-20, about 20-25, about 25-30, about 30-35, about 35-40, about 40-45, about 45-50, about 50-55, about 55-60, about 65-70, about 75-80, about 80-85, about 85-90, above about 90, or any combination of these ranges. In one embodiment the weight percent range of the water-soluble refractive index adjusting agent is about 47 to about 67. Examples of a suitable refractive index adjusting agent include 1,3-propanediol. Other water-soluble refractive index adjusting agent can include glycerol, 1,2-propanediol, propylene glycol), and hexylene glycol.

An optical interface medium with a suitable refractive index minimizes reflections at the window and skin interface. In some implementations, the refractive index of the interface medium is selected to correspond to the refractive index of skin or a portion of the applicator device. In various implementations, the refractive index range of the optical interface medium is about 1.33-1.5, about 1.33-1.34, about 1.34-1.35, about 1.35-1.36, about 1.36-1.37, about 1.37-1.38. about 1.38-1.39, about 1.39-1.40, about 1.40-1.41, about 1.41-1.42, about 1.42-1.43, about 1.43-1.44, about 1.44-1.45, about 1.45-1.46, about 1.46-1.47, about 1.47-1.48, about 1.48-1.49, about 1.49 to about 1.5, or any combination of these ranges. In one embodiment the refractive index range of the optical interface medium is about 1.33 to about 1.43.

The optical interface medium should have a suitable viscosity for easy application and removal. In some implementations, the interface medium includes a gelling agent at a range sufficient to cause the interface medium to gel, or to have a viscosity within a desired range. In various implementations, the weight percent range of the gelling agent in the interface medium is about 0.01-1, 1-2, about 2-3, about 3-4, about 4-5, about 5-6, about 6-7, about 7-8, about 8-9, about 9-10, about 10-15, about 15-20, about 20-25, about 25-30, about 30-35, about 35-40, about 40-45, about 45-50, about 50-55, about 55-60, about 60-65, about 65-70, about 70-75, about 75-80, about 80-85, about 85-90, above about 90, or any combination of these ranges. In one embodiment the weight percent of the gelling agent is about 0.05-3. An ideal viscosity range allows for easy movement of an EMR applicator device over a skin surface such that an appropriate irradiation time (dwell time) can be achieved. Examples of suitable gelling agents include a hydroxyethyl acrylate/sodium acryloyldimethyl taurate copolymer, a polyacrylate crosspolymer-6, an acrylic acid copolymer, an acrylates/C10-30 alkyl acrylate crosspolymer, a carbomer, and a sodium polyacrylate.

Exemplary viscosity ranges for the optical interface medium include about 50000-52000 cP, about 52000-54000 cP, about 54000-56000 cP, about 56000-58000 cP, about 58000-60000 cP, about 60000-62000 cP, about 62000-64000 cP, about 64000-66000 cP, about 66000-68000 cP, about 68000-70000 cP, about 70000-72000 cP, about 72000-74000 cP, about 74000-76000 cP, about 76000-78000 cP, about 78000-80000 cP, about 80000-82000 cP, about 82000-84000 cP, about 84000-86000 cP, about 86000-88000 cP, about 88000-90000 cP, about 92000-94000 cP, about 94000-96000 cP, about 96000-98000 cP, about 98000-100000 cP, about 100000-105000 cP, about 105000-110000 cP, about 110000-115000 cP, about 115000-120000 cP, about 120000-125000 cP, about 125000-130000 cP, about 130000-135000 cP, about 135000-140000 cP, about 140000-145000 cP, about 145000-150000 cP, about 150000-160000 cP, about 160000-170000 cP, about 170000-180000 cP, about 180000-190000 cP, about 190000-200000 cP, above about 200000 cP, or any combination of these ranges. In one embodiment the viscosity range for the optical interface medium is about 5000 to about 100000 cP, about 5000 to about 80000 cP, and in some embodiments in the range of about 5000 to about 8000 cP. In one embodiment, the viscosity ranges are as measured by the Brookfield method: RV spindle 27 and speed 30 rpm. In another embodiment, the viscosity ranges are as measured by the Brookfield method: LV, spindle 16, speed 1 rpm. Viscosity measurements can be made at accepted temperature ranges, including room temperature and 40 degrees C.

Ideal interface media are optically clear, at least within a wavelength range of interest, i.e., a range of treatment wavelengths. Optical clarity is related to both absorbance and scattering of incident light, and may depend on the wavelength of the light. In some preferred embodiments, the interface medium is optically clear across one or more of the following wavelength ranges: about 380-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1050 nm, about 1050-1100 nm, about 1100-1150 nm, about 1150-1200 nm, about 1250-1300 nm, about 1300-1350 nm, about 1350-1400 nm, about 1400-1450 nm, about 1450-1500 nm, about 1500-1550 nm, about 1550-1600 nm, about 1600-1650 nm, about 1650-1700 nm, about 1700-1750 nm, about 1750-1800 nm, about 1800-1850 nm, or about 1850-1900 nm. In one embodiment, the interface medium is optically clear across a wavelength range of about 380 nm to about 1600 nm. For one or more of these wavelength ranges, the interface medium is optically clear such that absorbance and optical scattering are each below selected values. For example, various implementations have absorbance below about 0.1 cm⁻¹, about 0.2 cm⁻¹, about 0.3 cm⁻¹, about 0.4 cm⁻¹, about 0.5 cm⁻¹, about 0.6 cm⁻¹, about 0.7 cm⁻¹, about 0.8 cm⁻¹, about 0.9 cm⁻¹ or 1 cm⁻¹; and optical scattering independently below about 0.1 cm⁻¹, about 0.2 cm⁻¹, about 0.3 cm⁻¹, about 0.4 cm⁻¹, about 0.5 cm⁻¹, about 0.6 cm⁻¹, about 0.7 cm⁻¹, about 0.8 cm⁻¹, about 0.9 cm⁻¹ or about 1 cm⁻¹ for one or more of wavelength ranges above.

The optical scattering of the gel is below about 1 cm⁻¹. In one embodiment the absorbance of the gel is below about 15 cm⁻¹, such as about 5 -15 cm⁻¹, and in one embodiment about 13 cm⁻¹. Where materials exhibit absorbance in the range of about 5 -15 cm⁻¹ the effective absorbance will be influenced by the thickness of a layer of gel on tissue. The absorption value relates the absorbance of the gel itself to the thickness of the gel as it is a measure of the percent of laser energy that is absorbed by the gel without passing through the gel to tissue. That is, if 98% of the laser energy passes through the gel without absorption, the absorption value is 2%.

The gel described herein should be of such a composition and should be applied to skin in a thin layer so as to yield low absorption. By way of example, the thickness of the gel described herein should be applied to tissue in a layer that is less than about 100 microns, and typically less than about 20 microns, and generally about 10 microns. The absorption of the gel described herein should be less than about 15%, such as less than about 5%, and typically about 2%. At a gel thickness of about 10 microns, for example, an exemplary gel having an absorbance of about 13 cm⁻¹ will allow about 98.7% of the laser energy to pass through the tissue, thus exhibiting an absorption of about 1.3%. If the gel thickness is as high as 100 microns the absorption will be about 13%.

As some dermatological phototherapeutic devices use high power laser irradiation, photostability is an important feature to consider when selecting an optical interface medium. The ideal optical interface medium exhibits photostability such that optical clarity is maintained upon exposure to EMR. For example, various implementations of interface media are photostable such that the can be exposed to EMR within a selected wavelength range up to about 10⁵, about 10⁶, about 10⁷, about 10⁸, about 10⁹, or about 10¹⁰ W/cm² without plasma formation, cavitation, or burning. The selected wavelength range can be about 380-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1050 nm, about 1050-1100 nm, about 1100-1150 nm, about 1150-1200 nm, about 1200-1250 nm, about 1250-1300 nm, about 1300-1350 nm, about 1350-1400 nm, about 1400-1450 nm, about 1450-1500 nm, about 1500-1550 nm, about 1550-1600 nm, about 1600-1650 nm, about 1650-1700 nm, about 1700-1750 nm, about 1750-1800 nm, about 1800-1850 nm, about 1850-1900 nm, or any combination of these wavelength ranges. In one example a focal spot width is about 20 microns and has a power output P of a single EMR source (e.g., a laser diode) in the range of about 15 mW and 1500 mW. A dwell time and power level can be selected to achieve a level of local fluence in the focal region in the range of about 10-1000 J/cm². Some implementations use pulsed irradiation while other implementations use continuous irradiation.

As the optical interface medium is used in contact with a patient's skin, possible interactions with the skin must be considered. Proper selection of the pH of the optical interface medium allows for avoidance of irritation of the skin and reaction with the EMR applicator device. Example pH ranges for the optical interface medium include about 4.5-4.6, about 4.6-4.7, about 4.7-4.8, about 4.8-4.9, about 4.9-5.0, about 5.0-5.1, about 5.1-5.2, about 5.2-5.3, about 5.3-5.4, about 5.4-5.5, about 5.5-5.6, about 5.6-5.7, about 5.7-5.8, about 5.8-5.9, about 5.9-6.0, about 6.0-6.1, about 6.1-6.2, about 6.2-6.3, about 6.3-6.4, about 6.4-6.5, about 6.5-6.6, about 6.6-6.7, about 6.7-6.8, about 6.8-6.9, about 6.9-7.0, or any combination of these ranges. In one embodiment the pH is in the range of about 5 to about 6.

Some optical interface media additionally include a numbing agent. Exemplary numbing agents are well known to those skilled in the art and include, but are not limited to, procaine, chloroprocaine, lidocaine, prilocaine, tetracaine, bupivacaine, cinchocaine, ropivacaine, benzocaine, dibucaine, and dimethocaine. In various implementations, the weight percent range of the numbing agent in the interface medium is in a pharmaceutically acceptable range such as about 1-2, about 2-3, about 3-4, about 4-5, about 5-6, about 6-7, about 7-8, about 8-9, about 9-10, about 10-15, about 15-20, about 20-25, about 25-30, about 30-35, about 35-40, about 40-45, about 45-50, about 50-55, about 55-60, about 60-65, about 65-70, about 70-75, about 75-80, about 80-85, about 85-90, above about 90, or any combination of these ranges.

Some optical interface media additionally include a soothing agent. Exemplary soothing agents are well known to those skilled in the art and include, but are not limited to, allantoin. In various implementations, the weight percent range of the soothing agent in the interface medium is in a pharmaceutically acceptable range such as about 1-2, about 2-3, about 3-4, about 4-5, about 5-6, about 6-7, about 7-8, about 8-9, about 9-10, about 10-15, about 15-20, about 20-25, about 25-30, about 30-35, about 35-40, about 40-45, about 45-50, about 50-55, about 55-60, about 60-65, about 65-70, about 70-75, about 75-80, about 80-85, about 85-90, above about 90, or any combination of these ranges.

An exemplary interface medium includes water at about 41.21 weight percent, 1,3-propanediol as a water-soluble refractive index adjusting agent at about 57.29 weight percent, and hydroxyethyl acrylate/sodium acryloyldimethyl taurate comonomer as a gelling agent at about 0.8 weight percent. This combination forms a single-phase transparent gel. The optical interface medium is optically clear, has a refractive index in the range of about 1.33 to about 1.43, and exhibits photostability upon exposure to electromagnetic radiation up to about 10⁸ W/cm² across a wavelength range from about 380 nm to about 1600 nm without plasma formation, cavitation, or burning. The exemplary optical interface media also include one or more antioxidants, antimicrobials, soothing agents, fragrance masking agents, or preservatives.

In some embodiments, the present disclosure encompasses methods of applying electromagnetic radiation (EMR) to skin tissue. An exemplary method includes applying a coupling medium to a target region of the skin; positioning an EMR delivery device in contact with the coupling medium overlaying the target region while moving the device over the skin; and applying EMR through the device as it is moved over the target region while the coupling medium maintains photostability without exhibiting plasma formation, cavitation, or burning. The coupling medium of this example is optically clear, with a refractive index in the range of about 1.33 to about 1.43, and a viscosity in the range of about 60000-100000 Cp (Brookfield method: LV, spindle 16, speed 1 rpm). In some embodiments the viscosity can be in the range of about 5000 to about 8000 cP (Brookfield method: RV spindle 27, speed 30 rpm).

Bubbles within the optical interface medium negatively affect optical transmission through the material. The EMR delivery device ideally is moved over the skin without bubble formation in the coupling medium, provided the coupling medium has a suitable coefficient of friction and other properties. Preparation of the optical interface medium by a process which minimizes air inclusion within the formulation can assist in the prevention of bubble generation during use of the optical interface medium. An ideal formulation has an air content low enough to avoid bubble generation during use. The coupling medium exhibits photostability upon exposure to electromagnetic radiation within a desired treatment range. In an example, the coupling medium exhibits photostability upon exposure to electromagnetic radiation up to about 10⁸ W/cm² without plasma formation, cavitation, or burning. The exemplary coupling medium maintains photostability to retain optical clarity such that across a wavelength range of about 380 to about 1600 nm absorbance is below about 1 cm⁻¹; and optical scattering is below about 1 cm⁻¹.

### EXAMPLES

### Example A

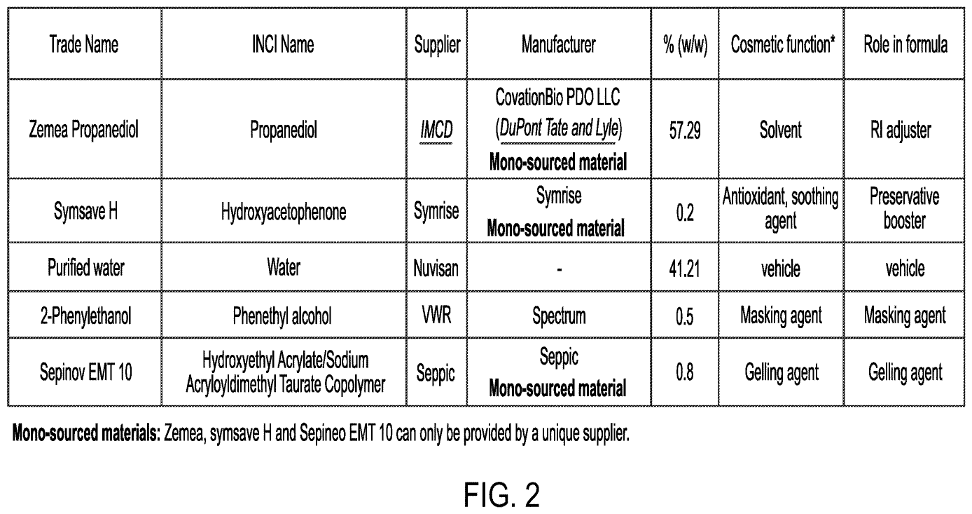

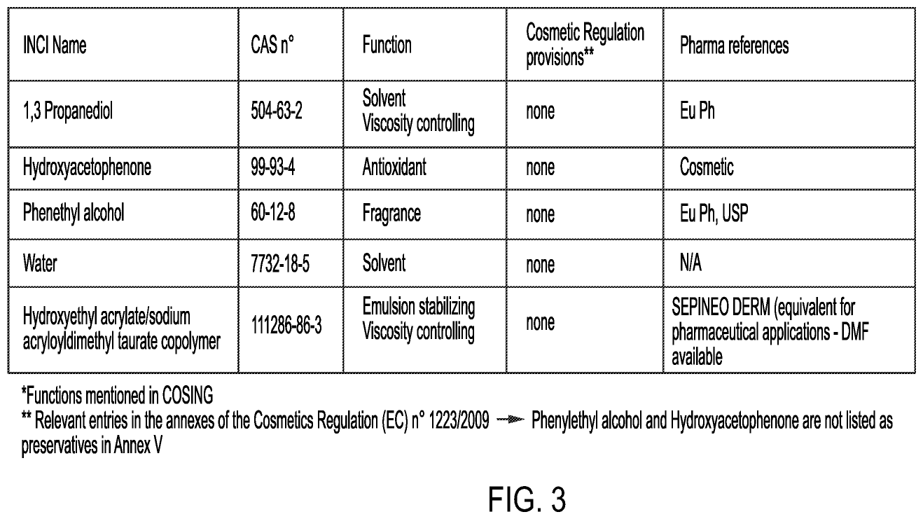

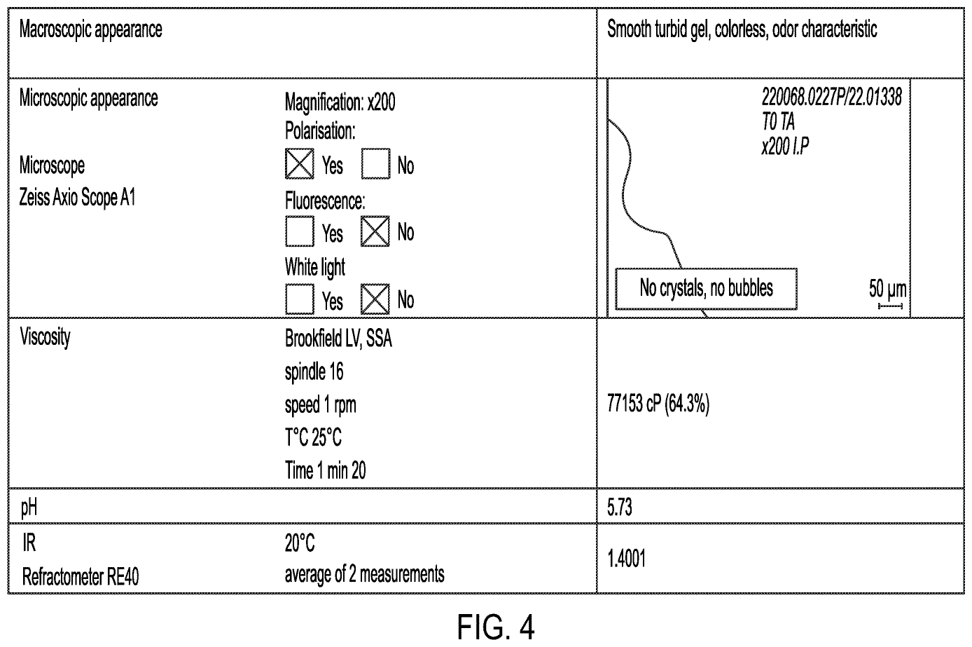

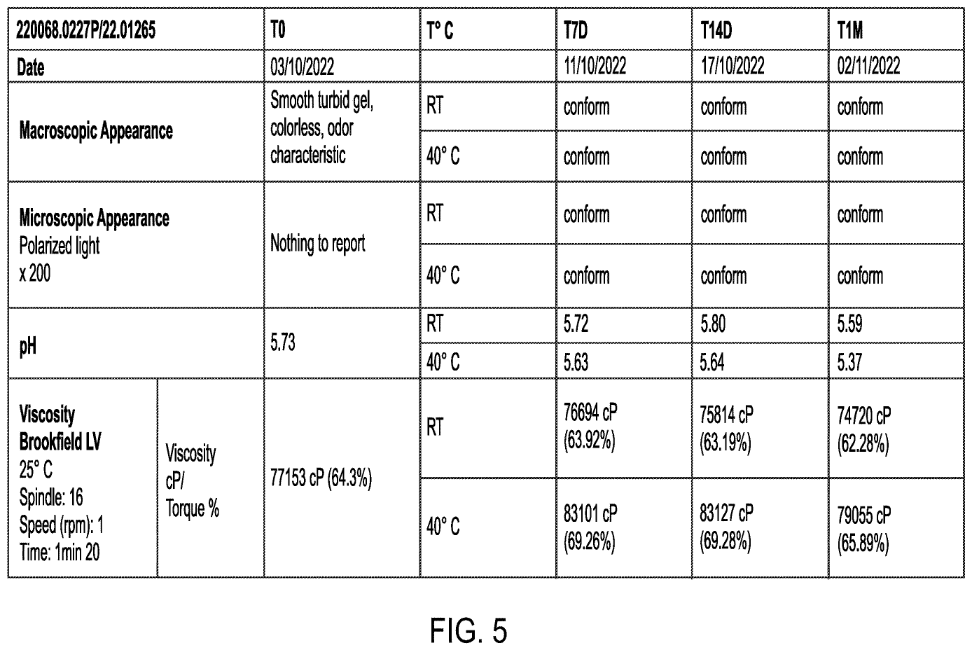

Example A demonstrated positive preliminary results in stability and performance assessments. The quantitative and qualitative composition of this formula is presented in FIG. 2 and raw material suppliers, functions and regulatory aspects are described in FIG. 3. Initial characteristics are provided in FIG. 4 and preliminary stability data after storage for 1 month at RT and 40° C. summarized in FIG. 5. This formula also passed USP and Eur Ph preservative efficacy testing criteria.

### Excipient Selection Rationale

Example A includes the following excipients:
Purified water: 41.21% w/w (Refractive index~1.333 at 20° C.)
Zemea (1,3-Propanediol): Added to the aqueous phase in a relatively large quantity (57.29% w/w) to increase the refractive index and facilitate the glide of the device head on the skin.
Sepinov EMT 10 (Hydroxyethyl Acrylate/Sodium Acryloyldimethyl Taurate Copolymer)
at 0.8% w/w: A universal, thickening-stabilizing-texturizing powder copolymer used to increase the viscosity of the product and provides a translucent appearance. This gelling agent is very well adapted for increasing the viscosity of high glycol content media.
Symsave H (Hydroxyacetophenone) 0.2% w/w: A cosmetic ingredient with antioxidant effect as well as soothing and moisturizing properties. It is not listed as preservative in European cosmetics Annex V, but aids in microbial preservation.
Phenylethyl alcohol 0.5% w/w: A preservative to preventing microbial contamination that also possesses a pleasant odor (Rose scent) and can mask the odor of other raw materials.

### Laboratory Scale Manufacturing Process for Example A.1

The lab process consists of 4 principal steps that are performed at room temperature:

Step 1—Dissolve Symsave H in Zemea in the main beaker under “deflocculating”/dispersing stirring equipment at room temperature.

Step 2—Purified water, then Phenylethyl alcohol are added while stirring at RT. Stirring is maintained until the mixture is clear and homogeneous.

Step 3—SEPINEO EMT 10 is sprinkled above the vortex created by the mixing system in order to avoid lumps. Stirring is maintained for a few minutes until the gel is viscous and homogeneous.

Step 4—Deaeration of the bulk after the manufacturing under vacuum.

### Scale up to 10 Kg

Larger batches of material were produced at 10 kg scale using a Becomix RW 15 CD equipped with a principal tank and a melting tank.

The main tank is equipped with a planetary blade with scraper and counter blade as well as a rotor-stator at the base to ensure effective mixing homogenization and product recirculation. This tank is equipped with a double heating jacket and can be used under vacuum.

The melting vessel is equipped with a mixing blade at the bottom and has a double heating jacket.

### Modification of the Lab Process

Two elements were modified at 10 kg scale:

### Addition of the Gelling Agent (Equivalent to Step 3 of the Lab Process)

Sepinov EMT 10 is wetted and pre-dispersed using a dispersing blade into a portion of the Zemea (7.29%) in a separate vessel. Subsequently, this mixture is introduced into the main tank by aspiration via the rotor-stator at the bottom of the tank while mixing with the planetary blade with scraper and counter blade.

### Optimization of Mixing Parameters

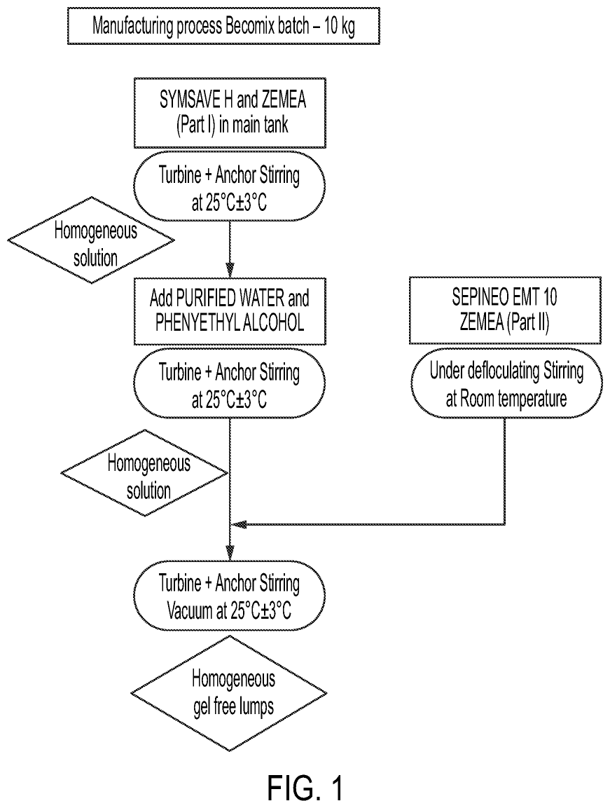

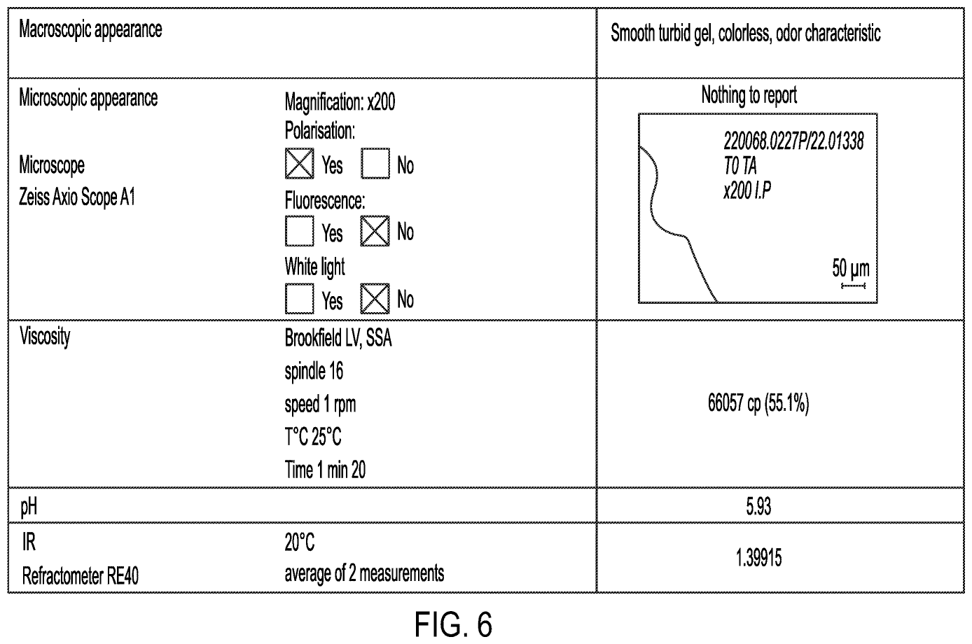

Terminal step of mixing under vacuum as described in FIG. 1. This resulted in Example A.2 (see FIG. 6 initial [T=0] characterization). Unfortunately, process modification did not address the problem of excess bubbles in the product. Having a bubble free coupling agent can be crucial to ensure appropriate imaging with the laser treatment device.

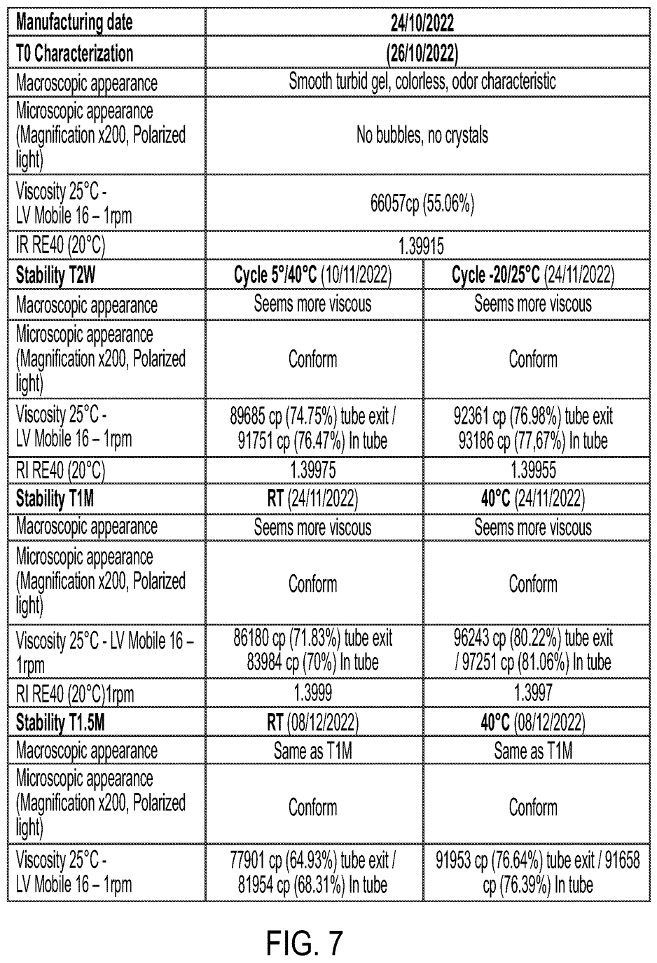

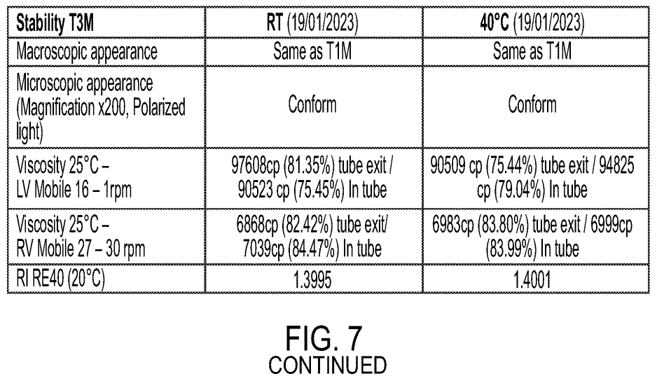

Despite the issues associated with air bubbles, Example A.2 appeared to possess encouraging stability data (FIG. 7). However, an increase in viscosity was observed after the initial interval. This may be related to lot-to-lot variation or hydration of the viscosity enhancing polymer (gelling agent).

### Initial Characteristics

Despite the modified process Example A.2 contained a lot of bubbles and had to be placed under vacuum to remove residual bubbles before T0 characterization (FIG. 6).

### R&D Physical Stability for 10 Kg Feasibility Batch

Physical stability of the hydrogel 10 kg feasibility batch packaged in 100ml HDPE tubes (Albea, Poland) is ongoing. Results of initial, T2W, T1M, T1.5M, and T3M intervals are presented in FIG. 7.

### Process Optimization to Avoid Bubbles (1 Kg)

Additional process options were evaluated to reduce the presence of air bubbles using a vacuum and Ika Magic Plant (2 kg) equipment at 1 kg scale.

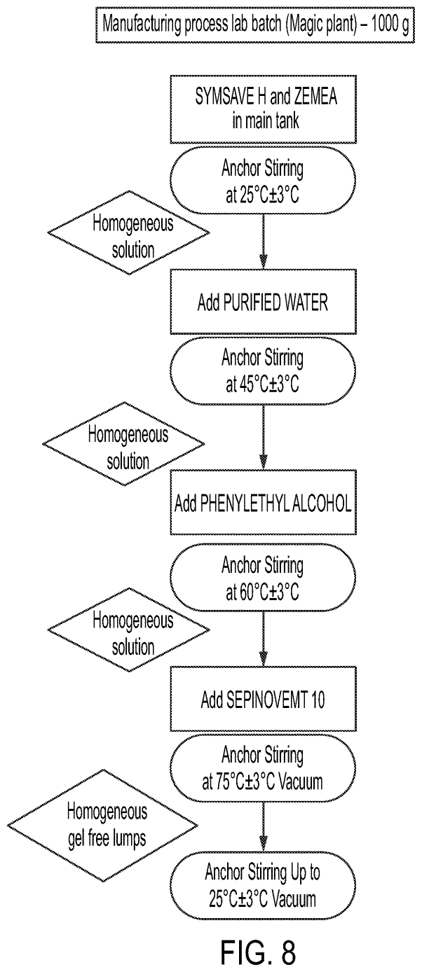

The process modifications included those described below and resulted in Example A.3, and the process is shown in FIG. 8:
Introduction of EMT10 into the main tank
   Direct addition into the water/Zemea phase charged into the main tank or
   Preparing a premix with Zemea in a secondary vessel
Preparation of a water gel composed of EMT10 and water followed by dilution with Zemea
Use of a hot process in order to reduce the viscosity of the gel while hot to facilitate air bubble evacuation under vacuum.

The process described above appeared to generate a product with acceptable characteristics, which was free from air bubbles.

### R&D Physical Stability for 10 Kg Feasibility Batch With Optimized Process to Avoid Air Bubbles—Example A.4

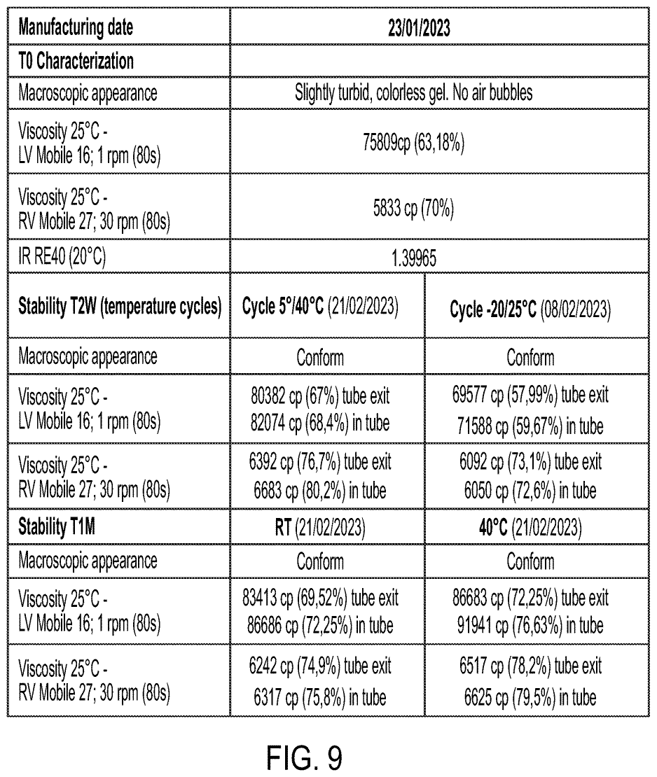

The process described in Example A.3 was applied to a 10 kg batch, and allowed the manufacturing of a batch free from air bubbles. As shown in FIG. 9, the batch demonstrated physical stability for one month, both at room temperature and at 40° C.

Approximating language, as used herein throughout the specification and claims, may be applied to modify any quantitative representation that could permissibly vary without resulting in a change in the basic function to which it is related. “Approximately,” “substantially,” or “about” can include numbers that fall within a range of 1%, or in some embodiments within a range of 5% of a number, or in some embodiments within a range of 10% of a number in either direction (greater than or less than the number) unless otherwise stated or otherwise evident from the context (except where such number would impermissibly exceed 100% of a possible value). Accordingly, a value modified by a term or terms, such as “about,” “approximately,” or “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or all of the group members are present in, employed in, or otherwise relevant to a given product or process. Furthermore, it is to be understood that the disclosed embodiments provide all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the disclosed embodiments where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the disclosed embodiments, or aspects of the disclosed embodiments, is/are referred to as comprising particular elements, features, etc., certain embodiments of the disclosure or aspects of the disclosure consist, or consist essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification. For example, any one or more active agents, additives, ingredients, optional agents, types of organism, disorders, subjects, or combinations thereof, can be excluded.

Where ranges are given herein, embodiments of the disclosure include embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise. It is also understood that where a series of numerical values is stated herein, the disclosure includes embodiments that relate analogously to any intervening value or range defined by any two values in the series, and that the lowest value may be taken as a minimum and the greatest value may be taken as a maximum. Numerical values, as used herein, include values expressed as percentages.

Although a few variations have been described in detail above, other modifications or additions are possible.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. An optical interface medium for dermatological phototherapy, comprising a single-phase, transparent, aqueous gel that is optically clear such that across a wavelength range of about 380 nm to about 1900 nm: absorbance is below 15 cm⁻¹; and optical scattering is below 1 cm⁻¹,
wherein the optical interface medium has a refractive index in the range of about 1.33 to about 1.5 with a viscosity in the range of about 5,000 to about 100,000 cP, and
wherein the optical interface medium exhibits photostability such that optical clarity is maintained upon exposure to electromagnetic radiation up to about 10⁸ W/cm² without plasma formation, cavitation, or burning.

2. The optical interface medium of claim 1, comprising 1,3-propanediol present at a range of about 5 to about 70 weight percent.

3. The optical interface medium of claim 1, further comprising:
water, present at a range of about 28.5 to about 93.5 weight percent;
a water-soluble refractive index adjusting agent, present at a range of about 5 to about 70 weight percent; and
a gelling agent, present at a range of about 0.05 to about 3 weight percent.

4. The optical interface medium of claim 1, wherein the viscosity of the optical interface medium is in the range of about 6,000 to about 8,000 cP as measured by the Brookfield method: RV, spindle 27 and speed 30 rpm.

5. The optical interface medium of claim 1, wherein the pH of the optical interface medium is in the range of about 5 to about 6.

6. The optical interface medium of claim 1, further comprising a numbing agent.

7. An optical interface medium for dermatological phototherapy, comprising:
water;
1,3-propanediol as a water-soluble refractive index adjusting agent; and
a gelling agent, configured to form a single-phase transparent gel with water and the refractive index adjusting agent;
wherein the optical interface medium is optically clear, has a refractive index in the range of about 1.33 to about 1.43, and exhibits photostability upon exposure to electromagnetic radiation up to about 10⁸ W/cm² without plasma formation, cavitation, or burning.

8. The optical interface medium of claim 7, wherein the gelling agent is a hydroxyethyl acrylate/sodium acryloyldimethyl taurate copolymer, a polyacrylate crosspolymer-6, an acrylic acid copolymer, an acrylates/C10-30 alkyl acrylate crosspolymer, a carbomer, or a sodium polyacrylate.

9. The optical interface medium of claim 7, further comprising one or more antioxidants, antimicrobials, soothing agents, fragrance masking agents, or preservatives.

10. The optical interface medium of claim 7, comprising about 28.5 to about 50 percent water by weight.

11. The optical interface medium of claim 7, comprising about 37 to about 70 percent refractive index adjusting agent by weight.

12. The optical interface medium of claim 7, comprising about 0.7 to about 1.1 percent gelling agent by weight.

13. A method of applying electromagnetic radiation (EMR) to skin tissue, comprising:
applying a coupling medium to a target region of the skin, the coupling medium being optically clear with a refractive index in the range of about 1.33 to about 1.43, and having a viscosity in the range of about 5,000 to about 8,000 cP as measured by the Brookfield method: RV, spindle 27 and speed 30 rpm;
positioning an EMR delivery device in contact with the coupling medium overlaying the target region while moving the device over the skin without bubble formation within the coupling medium; and
applying EMR through the device as it is moved over the target region while the coupling medium maintains photostability without exhibiting plasma formation, cavitation, or burning.

14. The method of claim 13, wherein the coupling medium exhibits photostability upon exposure to electromagnetic radiation up to about 10⁸ W/cm² without plasma formation, cavitation, or burning.

15. The method of claim 13, wherein the coupling medium maintains photostability to retain optical clarity such that across a wavelength range of about 380 to about 1600 nm: absorbance is below 15 cm⁻¹; and optical scattering is below 1 cm⁻¹.
